(19) United States
(12) Patent Application Publication
(10) Pub. No.: US 2007/0260192 A1
(43) Pub. Date: Nov. 8, 2007

(54) Safety syringe

(75) Inventors: Chien-Yuan Lee, Tamsui Town (TW); Chih-Cheng Kao, Taipei (TW)

Correspondence Address: KAMRATH & ASSOCIATES P.A., 4825 OLSON MEMORIAL HIGHWAY, SUITE 245, GOLDEN VALLEY, MN, 55422, US

(73) Assignee: Linfair Engineering (HK) Co., Ltd., Hong Kong (CN)

(21) Appl. No.: 11/418,969

(22) Filed: May 5, 2006

(51) Int. Cl. A61M 5/32

(52) U.S. Cl. 604/197

(57) ABSTRACT

A safety syringe has a barrel, a retractor, a needle hub and an outer sleeve. The barrel has a plunger mounted slidably in the barrel, a front end and a threaded protrusion formed on the end and has a through hole. The retractor is hollow, engages the threaded protrusion and has a first connector. The needle hub has an enlarged end having a needle longitudinally extending through the needle hub and a second connector corresponding to and connecting detachably with the first connector. The outer sleeve is mounted slidably around the needle hub, is mounted detachably and slidably around the retractor and the barrel to mount in detachably and has a front end, a cavity, an inner surface, a positioning protrusion and an annular protrusion mounted in the inner surface. The syringe is assembled easily and conveniently.

### BACKGROUND OF THE INVENTION

1. Field of Invention

The present invention relates to a safety syringe, and more particularly to a syringe that is assembled easily.

2. Description of the Related Art

A conventional syringe has an outer sleeve, a barrel and a needle hub. The outer sleeve has a rear open end and a front end having a through hole. The barrel selectively holds a liquid material such as water, liquid medication or the like and has an end having a threaded inner surface and a plunger. The plunger is slidably mounted in the barrel. The needle hub extends in the outer sleeve from the rear open end and has a threaded end and a needle. The threaded end screws in the threaded inner surface of the barrel. The needle passes through the outer sleeve from the rear open end and extends in the through hole of the outer sleeve. When the plunger is pushed, the material can be injected into a subject such as a human, an animal, an experimental material or other material.

However, when the syringe is assembled, a user takes time to screw the needle hub in the barrel. After the material is injected into the subject, the needle hub is disengaged from the barrel, so the used needle hub can be thrown away. Consequently, assembling the conventional syringe is very inconvenient for users.

To overcome the shortcomings, the present invention provides a safety syringe that is assembled easily and conveniently to mitigate or obviate the aforementioned.

### SUMMARY OF THE INVENTION

The primary objective of the present invention is to provide a safety syringe that is assembled easily and conveniently and prevents users from being pricked with a used needle.

To achieve the objective, a safety syringe has a barrel, a retractor, a needle hub and an outer sleeve. The barrel has a chamber to receive a liquid material, a plunger mounted slidably in the barrel, an end and a threaded protrusion formed on the end and has a through hole. The retractor is hollow, engages the threaded protrusion and has an outer surface and a first connector formed on the outer surface. The needle hub has an enlarged end having an outer diameter, a needle longitudinally extending through the needle hub and a second connector corresponding to and connecting detachably with the first connector. The outer sleeve is mounted slidably around the needle hub, is mounted detachably and slidably around the retractor and the barrel and has a front end, a cavity and an inner surface. The front end has a through hole to allow the needle to extend through the through hole. The inner surface has a positioning protrusion and an annular protrusion. The positioning protrusion is formed on the inner surface of the outer sleeve, is adjacent to the through hole and positions the enlarged end of the needle hub by stopping the enlarged end from moving. The annular protrusion is formed on the inner surface of the outer sleeve, is adjacent to the rear open end of the outer sleeve and has an inner diameter being smaller than the outer diameter of the enlarged end of the needle hub, so the needle hub can not detach from the outer sleeve.

Other objectives, advantages and novel features of the invention will become more apparent from the following detailed description when taken in conjunction with the accompanying drawings.

### BRIEF DESCRIPTION OF THE DRAWINGS

FIG. 1 is a partially exploded perspective view of a safety syringe in accordance with the present invention;

FIG. 2 is an exploded perspective view of the safety syringe in FIG. 1 with a chamber receiving a vial with a liquid material;

FIG. 3 is a top view in partial section of an embodiment of a first connector, a second connector and a retractor of the safety syringe in FIG. 1 with a first connector being a annular groove and a second connector being an annular protrusion before the needle piercing the vial;

FIG. 4 is an operational top view in partial section of the safety syringe in FIG. 1 after the safety syringe piercing the vial;

FIG. 5 is an operational top view in partial section of the safety syringe in FIG. 1 showing that the needle hub partially retracts into the outer sleeve;

FIG. 6 is an operational top view in partial section of the safety syringe in FIG. 5 showing that the needle hub totally retracts into the outer sleeve;

FIG. 7 is an operational top view in partial section of the safety syringe in FIG. 6 showing that the outer sleeve is disengaged from the barrel;

FIG. 8 is a partially enlarged top view in partial section of the safety syringe in with a retractor being cylindrical, a front end of a needle hub being tapered, the first connector being the annular protrusion and the second connector being the annular groove;

FIG. 9 is a partially enlarged top view in partial section of the safety syringe with the retractor being elongated and cylindrical and the needle hub being cylindrical; and

FIG. 10 is a top view in partial section of the safety syringe with a chamber to receive the liquid material.

### DETAILED DESCRIPTION OF THE INVENTION

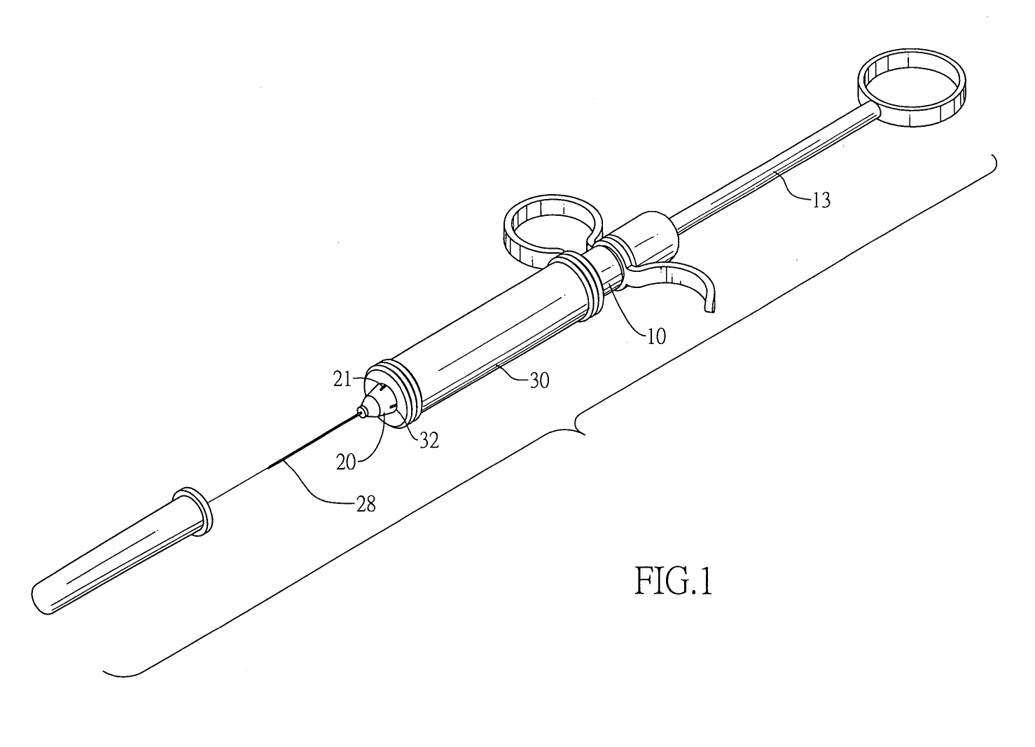

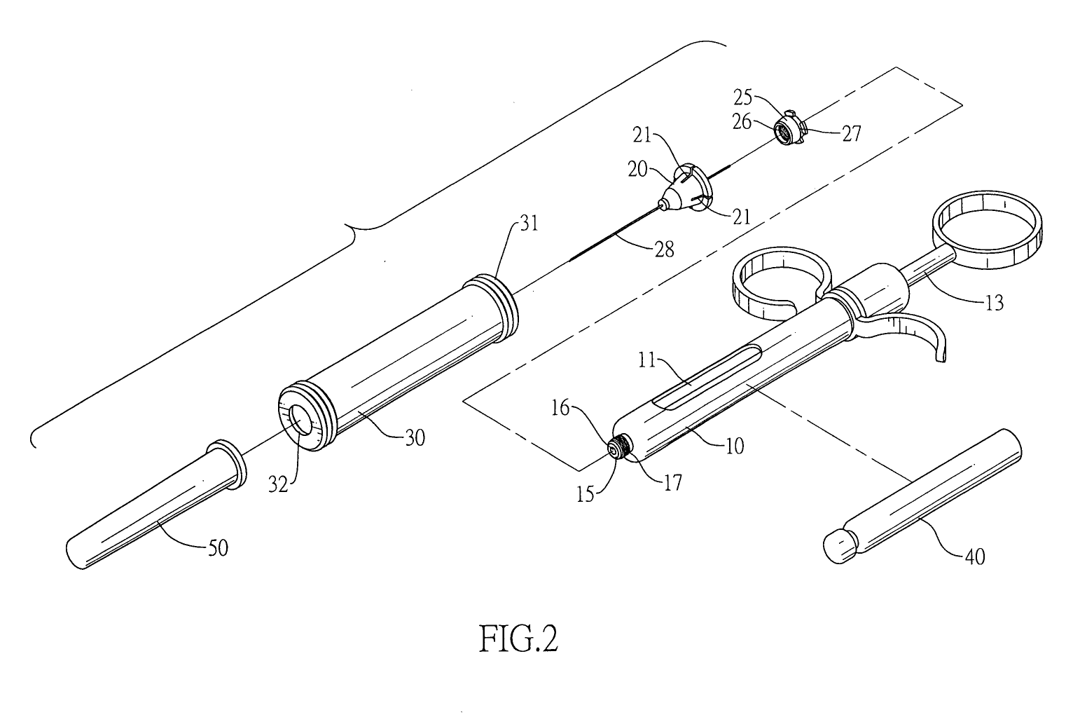

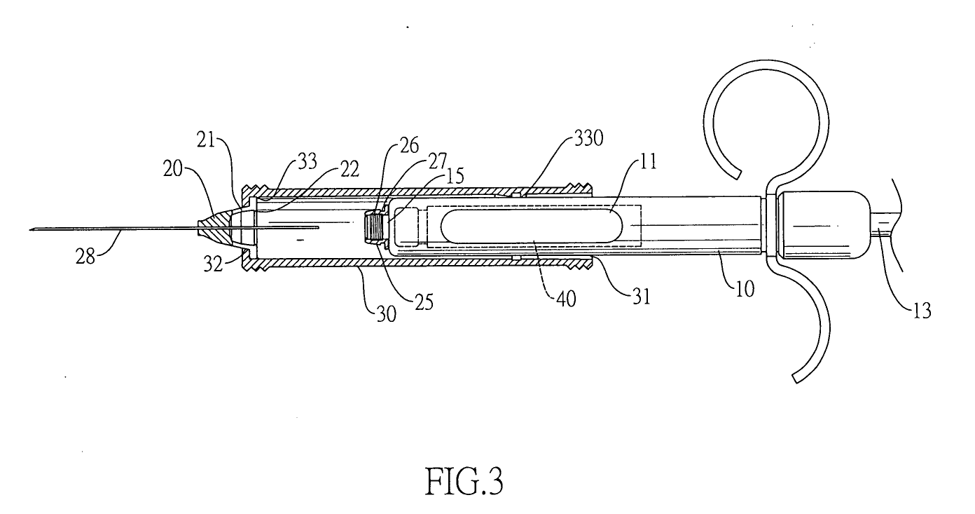

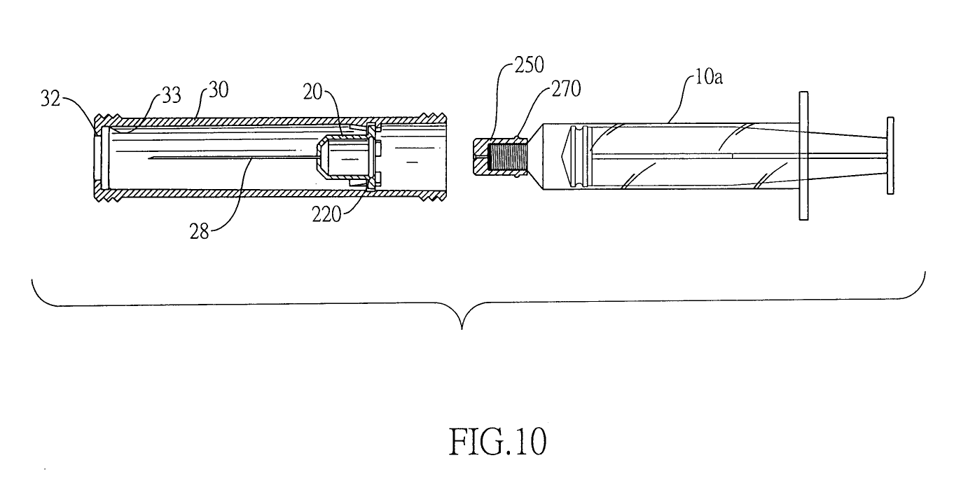

With reference to FIGS. 1 to 3 and 10, a safety syringe in accordance with the present invention has a barrel (10, 10a), a retractor (25, 250), a needle hub, an outer sleeve (30) and a cover (50).

The barrel (10, 10a) may receive and hold a vial (40) with a liquid material or the liquid material without any vial (40) and has an rear open end, a chamber, a front end, a threaded protrusion (15) and an outer surface and a plunger (13). The liquid material may be water, liquid medication or the like. The chamber is defined in the barrel (10, 10a), communicates with the rear open end and may receive the vial (40) with the liquid material or receive the liquid material. The threaded protrusion (15) is formed on the front end and has an outer surface and a through hole (16). The outer surface has an outer thread (17) formed on the outer surface. The through hole (16) is axially formed in the threaded protrusion (15) and corresponds to and communicates with the chamber. The outer surface may have an elongated recess (11) formed radially through the outer surface and communicating with the chamber so users are able to see the material in the vial through the elongated recess (11). The plunger (13) is mounted movably in the barrel (10, 10a) from the rear open end.

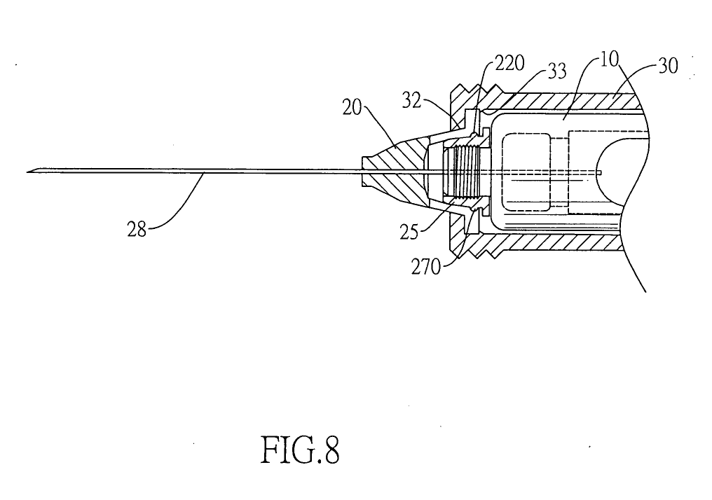

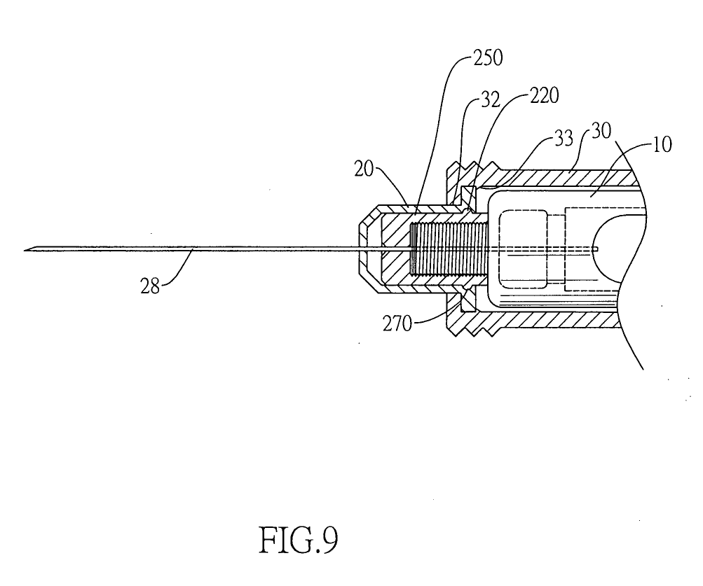

With further reference to FIGS. 8 and 9, the retractor (25, 250) is hollow, may be cylindrical or elongated, is engaged firmly with the threaded protrusion (15), and has an inner surface, an inner thread (26), an outer surface and a first connector (27, 270). The inner thread (26) are formed in the inner surface and corresponds to the outer thread (17) of the threaded protrusion (15) to combine the retractor (25, 250) with the threaded protrusion (15). The first connector (27, 270) is formed on the outer surface of the retractor (25, 250) and may be an annular protrusion or an annular groove.

The needle hub detachably connects with the retractor (25, 250), may be tapered or cylindrical and has a body (20) and a needle (28). The body (20) has a front end, an enlarged end, an inner surface, a second connector (22, 220) and a mounting hole. The front end may be tapered. The enlarged end has an outer diameter, multiple recesses (21) formed radially in the enlarged end at intervals, so the enlarged end is able to expand. The second connector (22, 220) is formed in the inner surface of the body (20), corresponds to the first connector (27, 270), detachably connects with the first connector (27, 270) and may be an annular protrusion when the first connector (27, 270) is the annular groove or an annular groove when the first connector (27, 270) is the annular protrusion. The mounting hole is formed through the body (20). The needle (28) is mounted firmly in and longitudinally extends through the mounting hole of the body (20) and may protrude in chamber in the barrel (10, 10a) through the through hole (16) of the threaded protrusion (15).

The outer sleeve (30) is mounted slidably around the needle hub, is mounted detachably and slidably around the retractor (25, 250) and the barrel (10, 10a) and has a cavity, a rear open end, a front end and an inner surface. The cavity is defined in the outer sleeve and receives the needle hub, the retractor (25, 250) and the barrel (10, 10a). The rear open end allows the needle hub, a retractor (25, 250) and the barrel (10, 10a) to slidably mount in the cavity in the outer sleeve (30). The front end has a through hole (32) defined through the front end to allow the needle (28) and the front end of the body (20) of the needle hub to extend through the through hole. The inner surface has a positioning protrusion (33) and an annular protrusion (330). The positioning protrusion (33) is formed on the inner surface of the outer sleeve (30), is adjacent to the through hole (32) and positions the enlarged end of the needle hub (20) by stopping the enlarged end from moving. The annular protrusion (330) is formed on the inner surface of the outer sleeve (30), is adjacent to the rear open end of the outer sleeve (30) and has an inner diameter being smaller than the outer diameter of the enlarged end of the body (20), so the needle hub can not detach from the outer sleeve (30).

The cover (50) covers the needle (28) and the front end of the body (20) to keep the needle clean before the needle (28) is used.

Before the material in the safety syringe is injected into a subject such as a human, an animal, an experimental material or other material, the needle hub is mounted in the front end of the outer sleeve (30) and is positioned by the positioning protrusion (33) and the needle (28) and the front end of the needle hub are covered by the cover (50). This time, the retractor (25, 250) firmly engages the threaded protrusion (15) of the barrel (10, 10a) and the plunger (13) is mounted slidably in the barrel (10, 10a).

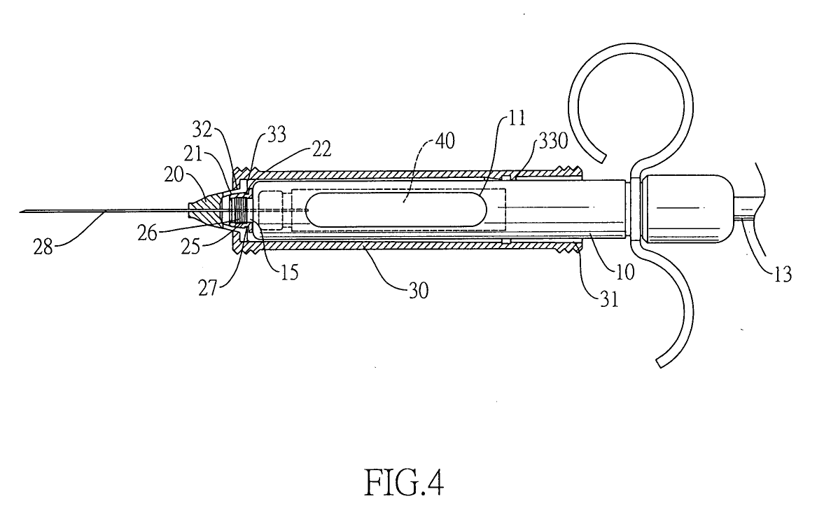

With further reference to FIG. 4, when the material in the vial (40) is injected into the subject, the vial (40) is mounted in the barrel (10, 10a). The barrel (10, 10a) and retractor (25, 250) is slidably mounted in the outer sleeve (30) and the retractor (25, 250) connects detachably with the body (20) of the needle hub by the first connector (27, 270) and the second connector (22, 220). In the mean time, the needle (28) that is mounted in the outer sleeve (30) pierces in the vial (40) through the retractor (25, 250), the through hole of the threaded protrusion (15) and the hole of the barrel (10, 10a). Finally, the plunger (13) is pushed and the material is injected into the subject.

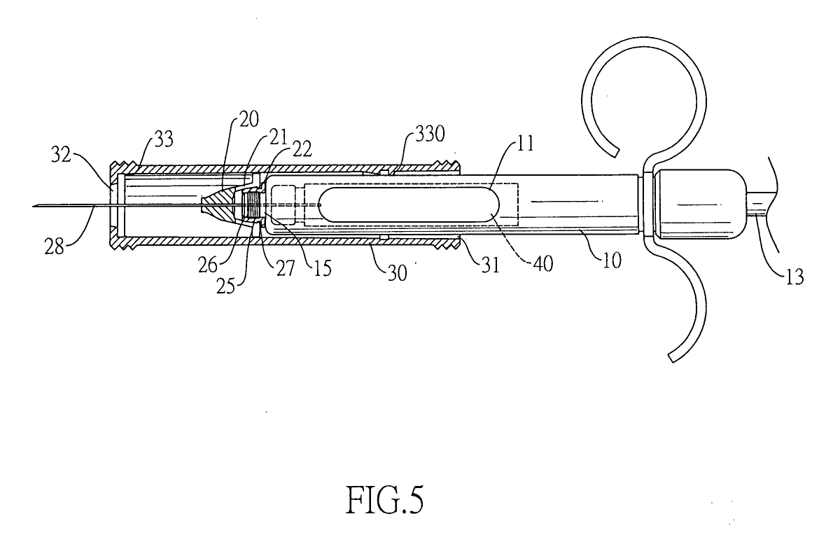

With further reference to FIG. 5, after the material in the vial (40) is injected into the subject, the barrel (10, 10a) with the retractor (25, 250) is pulled out of the outer sleeve (30). The needle (28) and the needle hub are pulled by the barrel (10, 10a), slide and retract into the outer sleeve (30). Because the first connector (27, 270) on the retractor (25, 250) firmly connects to the second connector (22, 220) of the needle hub, the positioning protrusion (33) in the outer sleeve (30) can not stop the needle hub (20) pulled by the barrel (10a).

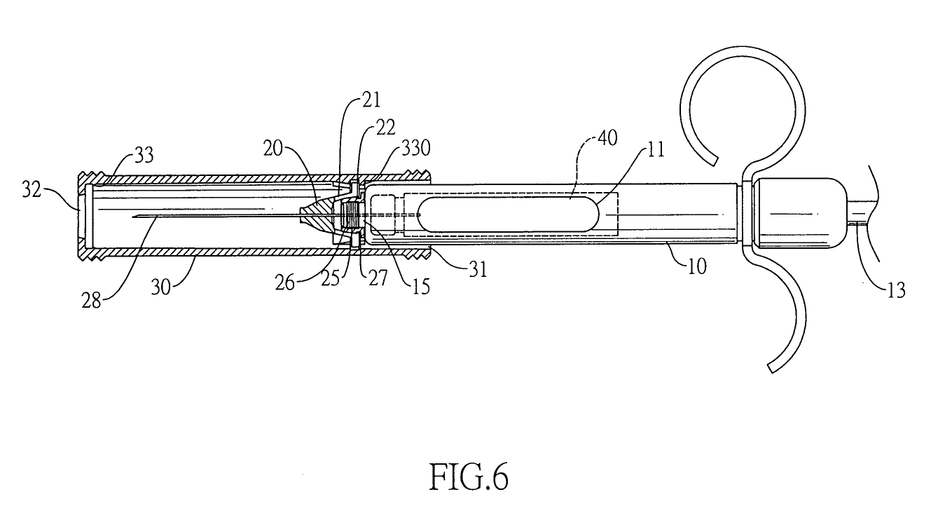

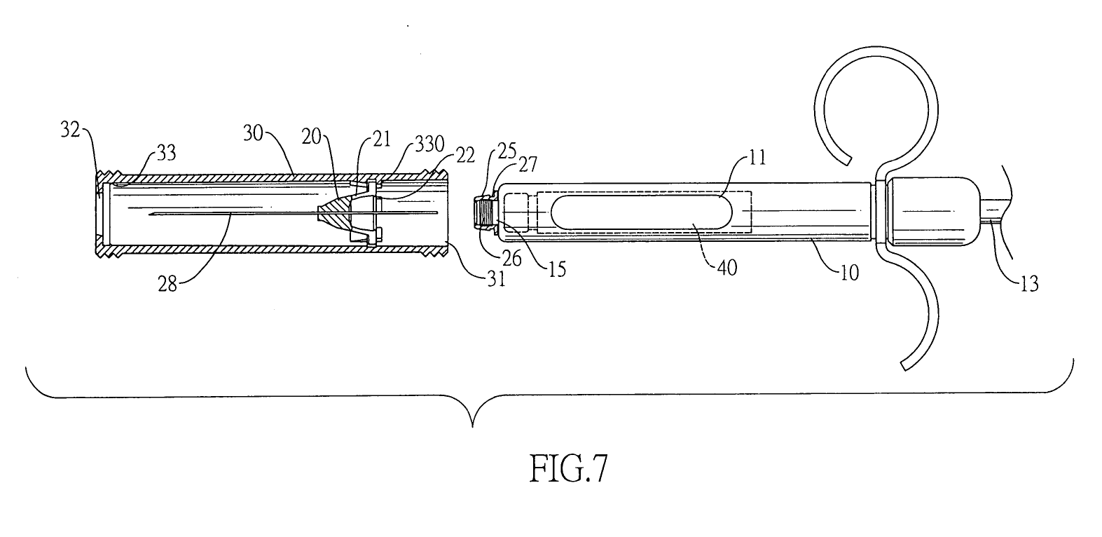

With further reference to the FIGS. 6 and 7, when the needle (28) totally moves into the outer sleeve (30) and the needle hub reaches the annular protrusion (330). Because the inner diameter of the annular protrusion (330) is smaller than the outer diameter of the enlarged end of the body (20), the needle hub can not be detached from the outer sleeve (30). The enlarged end of the needle hub can be expanded because of the recesses (21) of the body (20) to allow the second connector (22, 220) of the body (20) to be detached from the first connector (27, 270) of the retractor (25, 250). When the barrel (10, 10a) and the retractor (25, 250) are detached from the outer sleeve (30), the needle hub still stays in the outer sleeve. The outer sleeve (30) holding the needle (28) inside will be thrown out to prevent from piercing human's body.

The structure of the syringe is simple. Therefore, the safety syringe is assembled easily and conveniently for users to use.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A safety syringe comprising:
a barrel having
a rear open end;
a chamber defined in the barrel and communicating with the rear open end;
a front end;
a plunger mounted movably in the chamber in the barrel;
a threaded protrusion formed on the front end and having a through hole axially formed in the threaded protrusion;
a retractor being hollow and firmly engaged with the threaded protrusion and having
an outer surface; and
a first connector formed on the outer surface of the retractor;
a needle hub detachably connecting with the retractor and having
a body having
an outer diameter;
an inner surface;
a second connector formed in the inner surface of the body, corresponding to the first connector and detachably connecting with the first connector; and
a mounting hole formed through the body;
a needle mounted firmly in and longitudinally extending through the mounting hole of the body; and
an outer sleeve mounted slidably around the needle hub to mount slidably in, mounted detachably and slidably around the retractor and the barrel and having
a cavity defined in the outer sleeve and receiving the needle hub, the retractor and the barrel;
a rear open end;
a front end having a through hole defined through the front end and allowing the needle to extend through the through hole; and
an inner surface having
a positioning protrusion formed on the inner surface of the outer sleeve, adjacent to the through hole of the front end and positioning the needle hub; and
an annular protrusion formed on the inner surface of the outer sleeve, adjacent to the rear open end of the outer sleeve and having an inner diameter being smaller than the outer diameter of the body.

2. The safety syringe as claimed in claim 1, wherein the body of the needle hub further has an enlarged end corresponding to the retractor and having multiple recesses formed radially in the enlarged end at intervals.

3. The safety syringe as claimed in claim 1, wherein:
the first connector is an annular protrusion; and
the second connector is an annular groove corresponding to and connecting with the annular protrusion.

4. The safety syringe as claimed in claim 2, wherein:
the first connector is an annular protrusion; and
the second connector is an annular groove corresponding to and connecting with the annular protrusion.

5. The safety syringe as claimed in claim 1, wherein:
the first connector is an annular groove; and
the second connector is an annular protrusion corresponding to and connecting with the annular groove.

6. The safety syringe as claimed in claim 2, wherein:
the first connector is an annular groove; and
the second connector is an annular protrusion corresponding to and connecting with the annular groove.
